(19) United States
(12) Patent Application Publication
(10) Pub. No.: US 2020/0365267 A1
(43) Pub. Date: Nov. 19, 2020

(54) DECENTRALLY CONTROLLED IMAGING-BASED PATIENT DATA EXTRACTION

(71) Applicant: Siemens Healthcare GmbH, Erlangen (DE)

(72) Inventor: Lars Lauer, Neunkirchen (DE)

(21) Appl. No.: 16/869,977

(22) Filed: May 8, 2020

(30) Foreign Application Priority Data
May 15, 2019 (EP) 19174633.8

(51) Int. Cl. G16H 40/67; G16H 30/20; G16H 30/40; G16H 50/20; G06N 3/08; G10L 15/22

(52) U.S. Cl. CPC G16H 40/67; G16H 30/20; G10L 15/22; G16H 50/20; G06N 3/08; G16H 30/40

(57) ABSTRACT

A decentrally controlled imaging method is provided. A selected control entity is connected by a data transmission network to a communication device of a medical technical imaging system. An image recording process of a field of view of a patient is remotely controlled by the control entity via the network-based communications link.

### CROSS REFERENCE TO RELATED APPLICATIONS

This patent application claims the benefit of EP 19174633.8 filed May 15, 2019, which is hereby incorporated in its entirety FIELD

Embodiments relate to a decentrally controlled imaging method, a diagnosis data extraction method, and a decentralized image data extraction system.

### BACKGROUND

Medical technical imaging systems are in many cases indispensable for making a diagnosis. However, for effective use of such devices, a large number of qualified people is required, that, for example, in less developed countries, are not always available. For example, qualified personnel are required for the preparation of an imaging process, for example, for defining an imaging protocol. A medical technical assistant (MTA) is conventionally available for this task. Qualified personnel, in other words a qualified radiologist, are also required for evaluation of the image data. If qualified personnel are scarce, then, conventionally, control and evaluation of the imaging are centralized. Between the imaging system and a virtual qualified operator there is a communications link, via which the imaging process may be monitored and controlled. However, even with such a solution as this there must always be enough qualified personnel available in the central institution. A similar problem also exists with the evaluation of the generated image data. For example, it would be desirable to be able to make a timely diagnosis on the basis of image data even during nighttime operation of a hospital. Usually, no radiologists are locally available for the diagnosis of the image data at nighttime, however.

There is therefore the problem of being able to carry out medical technical imaging methods and evaluations of the generated image data also independently of locally available, suitably qualified personnel.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

In the decentrally controlled imaging method, a control entity, that may be connected by a data transmission network to a communication device of a medical technical imaging system, is determined and selected. As a data transmission network, a combination of computer networks, such as the Internet, may be used. As a medical technical imaging system, for example, a CT device or an MR system may be used. A control entity refers to an institution, that for the operation of a medical technical imaging system may provide the necessary control information or control commands. This may include a communications interface for a medical technical assistant or an automated arithmetic unit, that includes an algorithm or program that follows the algorithm for controlling an imaging process. The algorithm may be generated on the basis of a model or based on artificial intelligence. A network-based communications link is initialized between the communication device of the medical technical imaging system and the control entity. An image recording process of a field of view of a patient is remotely controlled by the control entity via the network-based communications link. The control entity and the medical technical imaging institution may be located far apart from each other. The two units are connected together for the imaging process via the data transmission network. The control entity may be flexibly used for the remote control of medical technical institutions at different locations. Conversely, a user of a medical technical imaging institution may search via the data transmission network for a currently available control entity, whereby a high level of flexibility, efficiency and timely availability of a competent operation of a medical technical imaging system is made possible, even when, for example, no qualified personnel are available in situ, as is the case, for example, during the nighttime. For example, operation with nurses only and by a medical technical assistant available during the nighttime in a different time zone or with the aid of suitable control programs, that are implemented for example on the basis of a Cloud, is possible.

The decentrally controlled imaging method is carried out in the diagnosis data extraction method. Furthermore, a specialist workstation, for example a teleradiologist station, that may be connected on the basis of a network to the communication device of a medical technical imaging system, is determined and selected from a plurality of specialist workstations. The specialist workstation may include a specialist workstation for a qualified operator, what is known as a teleradiologist. The image data generated in the image recording process is transferred to the selected specialist workstation and diagnosis data is output by the specialist workstation. The diagnosis data may be sent from the specialist workstation to the communication device of the medical technical imaging system and to a user or a patient, alternatively to the patient at home. Like the control entity, the specialist workstation may also be automated, i.e. the image data may be processed by a computer program stored in the data transmission network.

The decentralized image data extraction system includes a medical technical imaging system. A switching unit for the determination and selection of a control entity, that may be connected via a data transmission network to a communication device of the medical technical imaging system is also part of the decentralized image data extraction system. The switching unit may be configured to be both hardware-based and software-based. For example, the switching unit may include a switching program, that switches a medical technical imaging institution to a control entity. The decentralized image data extraction system also includes an initialization device for initializing a network-based communications link between the communication device of the medical technical imaging system and the control entity. Furthermore, the decentralized image data extraction system includes a control entity for remotely controlling an image recording process of a field of view of a patient via the network-based communications link. The control entity may be located far away from the medical technical imaging system. The decentralized image data extraction system shares the advantages of the decentrally controlled imaging method.

The diagnosis data extraction system includes the decentralized image data extraction system. In addition, the diagnosis data extraction system includes a switching unit for network-based determination and selection of one specialist workstation, that may be connected to the communication device of a medical technical imaging system, from a plurality of specialist workstations. The specialist workstation includes a specialist workstation for a qualified operator. Part of the diagnosis data extraction system is also an initialization device for initializing a network-based communications link between the communication device of the medical technical imaging system and the selected specialist workstation for transferring the image data generated during the image recording process to the selected specialist workstation. The diagnosis data extraction system also includes an input interface for outputting diagnosis data from the specialist workstation. The diagnosis data extraction system shares the advantages of the diagnosis data extraction method.

In the method for the extraction of training data, that, for example, occurs using the decentrally controlled imaging method, an automated image data extraction system including a medical technical imaging system, a control entity, a communication device, and an automated control system is set up. The automated control system is used to control an imaging process of the medical technical imaging system. The automated image data extraction system may be the decentralized image data extraction system. In the method for the extraction of training data, the automated control system is temporarily replaced by a specialist workstation with a specialist in order to extract training data. In other words, to extract training data the automated operation is replaced by an operation with a specialist. Furthermore, within the framework of the method communication between the specialists and a user and/or the imaging medical technical system, that occurs during the course of implementation of a controlled imaging method, is logged. The communication may include speech, transfer of image data, control data, measurement data, etc., the data logged in the process is prepared. When preparing the data, for example a transformation into a processed format may occur. In addition, the logged data and/or prepared logged data is provided as training data. Training data includes what is known as input training data and output training data. It is used for training a neuronal network that is adapted to the collected training data. During the course of training a recursive process of adaptation to the existing training data takes place. Advantageously, the input and output data, that is collected anyway during imaging that is controlled by specialists, may be used for extensive flexibilization and automation of the imaging. For example, frequently required imaging processes or applications to particular body parts, provide correspondingly extensive data material, so the applications may be automated first of all or with the aid of training processes and neural networks.

In the method for providing a trained automated control system, labeled input training data is received. Furthermore, labeled output training data is received, wherein the output training data is connected with the input training data. Labeled training data should be taken to mean data that are functionally related to each other and are associated with each other. With the method for providing a trained automated control system, a function based on the input training data and the output training data is trained with the aid of an artificial neural network and the trained function is provided for an automated control system. The automated control system may be used for imaging control. The control of imaging processes may be automated and thus rendered independent of the availability of specialists.

The training data extraction system includes an automated image data extraction system. The automated image data extraction system includes a medical technical imaging system, a control entity, a communication device, and an automated control system. The automated image data extraction system may include the decentralized image data extraction system. The training data extraction system also includes a specialist workstation, that temporarily replaces the automated control system in order to extract training data. Also, part of the training data extraction systems is a logging unit for logging the communication between the specialists and a user and/or the imaging medical technical system. The training data extraction system also may include a data preparation unit for preparing the logged data. In addition, the training data extraction system includes an input interface for outputting the logged data and/or prepared data as training data. The training data extraction system shares the advantages of the method for extracting training data.

The training system includes a first training interface for receiving input training data. Part of the training system is a second training interface for receiving output training data. The output training data is connected with the input training data. The training system also includes a training arithmetic unit for training a function for automated control based on the input training data and the output training data. Part of the training system is a third training interface for providing the training function for an automated control system. The training system shares the advantages of the method for providing a trained automated control system.

The fundamental components of the systems may be configured for the most part in the form of software components. This relates to, for example, the switching unit, the initialization device and the control entity of the decentralized image data extraction system, the switching unit, the initialization device of the diagnosis data extraction system, the training arithmetic unit of the training system and the logging unit and the data preparation unit of the training data extraction system. The components may also partly, for example if fast calculations are involved, be implemented in the form of software-supported hardware, for example FPGAs or the like. Similarly, the required interfaces, for example if it is merely a matter of transfer of data from other software components, may be configured as software interfaces. However, they may also be configured as interfaces constructed in terms of hardware, that are controlled by suitable software.

An implementation largely in terms of software includes the advantage that even previously used data transfer networks may be easily retrofitted by way of a software update in order to operate. In this regard the object is also achieved by a corresponding computer program product with a computer program, that may be loaded directly into a storage device of one or more sub-systems of a data transfer network of this kind, including program segments to carry out the steps of the method that may be implemented by way of software if the program is run in the data transmission network. Apart from the computer program, a computer program product of this kind may include additional components, such as documentation and/or additional components, also hardware components, such as hardware keys (dongles, etc.) for using the software.

A computer-readable medium, for example a memory stick, a hard disk or another portable or permanently fitted data carrier, on which the program segments of the computer program, that may be read-in and executed by an arithmetic unit of the control device are stored, may be used for transportation to the sub-system and/or for storage on or in this sub-system. The arithmetic unit may have for this purpose, for example, one or more cooperating microprocessor(s) or the like. The arithmetic unit may be, for example, part of a terminal or a control device of an imaging system, such as an MR system or a CT system, but it may also be part of a remotely positioned server system within the data transfer network.

In one embodiment of the decentrally controlled imaging method, the control entity includes a specialist workstation, at which control data is acquired and transferred via the network-based communications link for logging creation and for carrying out the imaging method. Any remote imaging system may be associated with a specialist workstation occupied by a qualified person, so human capacities may be used more flexibly and effectively.

In one embodiment of the decentrally controlled imaging method, the image recording process is remotely controlled on the basis of a dialogue, conducted via the network-based communications link, between a qualified operator at the specialist workstation and a user situated at the location of the medical technical imaging system. In this variant, the user receives instructions for operation of the imaging institution that he uses from the qualified operator. The user may conduct communication on the basis of the instructions themselves in order to prepare imaging. Direct control of the imaging system from the specialist workstation is not necessary.

At least one of the qualified operators may be simulated by an automated control system trained with the aid of training data on the basis of artificial intelligence. The qualified operator may be replaced by an algorithm or a computer program that implements an algorithm, so increased operational readiness, lower costs, and improved availability of the medical technical services may be achieved.

Use of the trained automated control system may be started as a function of whether a sufficient number of training data sets are available for the training process of the respective automated control system. Automation of control of the imaging or diagnosis is concentrated on applications that are used most frequently and in addition, owing to the broadest data base, may also be most reliably automated.

Tasks relating to imaging or diagnosis are classified into tasks, that are suitable for a virtual qualified operator or an automated system, on the basis of artificial intelligence. In other words, even the decision itself as to whether the remote control of the imaging or the diagnosis is to occur by way of a person or automatically is made by a trained AI-based computer program (AI=abbreviation for artificial intelligence). This decision process may also be implemented independently of existing qualified personnel and in this way, personnel costs may be reduced and the availability of this decision process may be increased. Use of AI-based solutions may be made dependent for example on the respective country-specific legislation. For example, for an application of this kind an official approval of a particular examination is required for automated control. If this approval exists in a particular country then the respective examination may be performed on the basis of being in this country, while the same examination of a patient, who is situated in a country without approval, occurs for example with the aid of a virtual operator.

The medical technical imaging system of the decentralized image data extraction system may include a system of the following system types:

a CT system, an MR system.

The types of imaging systems require extensive knowledge during operation and control of the imaging processes. It is advantageous precisely in the operation of this type of systems if an operation may be enabled independently of locally available, suitable personnel.

In an embodiment of the method for the extraction of training data, the training data includes as input training data at least one of the following data types: patient data, task-specific data, for example examination data, technical data relating to the medical technical imaging system, scout image data, image data.

The input training data includes information, that is required as the basis for carrying out the respective subprocesses of an imaging process or diagnosis process.

Further data sources for input training data are for example: the patient anamnesis of the referring doctor and data on preliminary findings from the patient file or data of a patient-monitoring camera in the medical technical imaging system or from a ceiling camera in the examination space. This data may be used for the validation of other data, such as the patient's weight, the height, and the gender. Furthermore, the cameras may be used in situ in the case of use of an MR system for the examination for monitoring the correct positioning of receive coils by the operator.

As output training data the training data includes at least one of the following data types: control data, that was generated by specialists, for example operators and/or radiologists, the number of an organ program, alignment data, an instruction to repeat the image recording, adjusted parameter data for repeated image recording, and/or diagnosis data e.g. radiological data.

Further data sources for output training data are for example structural parameters, extracted from the image data, on image quality, such as the signal-to-noise ratio or the artifact strength of movement artifacts.

The output training data forms the respective result of a respective subprocess of an imaging process or diagnosis process. A subprocess may relate to the adaptation of the imaging system to an indication, the imaging planning on the basis of scout imaging, the actual imaging, the diagnosis process and a decision as to whether imaging should be repeated with a contrast medium in the course of what is known as a refinement scan.

In an embodiment of the diagnosis method, the automated control system is trained according to the method for providing a trained automated control system. The described training method may also be used for automation of the diagnosis. This kind of automation allows personnel to be reduced and the availability and reliability of the diagnosis to be increased.

Embodiments also relate to a training system, that includes a first training interface for receiving input training data, a second training interface for receiving output training data, the output training data connected with the input training data (TE), a training arithmetic unit for training a function for the automated control based on the input training data and the output training data, and a third training interface for providing the training function for an automated control system.

Furthermore, embodiments relate to a computer program product including a computer program, that may be loaded directly into a Cloud-based storage device, including program segments, in order to carry out all steps of a method when the computer program is run on the basis of a Cloud. Furthermore, embodiments relate to a computer-readable medium, on which program segments that may be read-in and executed by an arithmetic unit are stored in order to carry out all steps of a method when the program segments are run by the arithmetic unit.

The training system, the computer program product and the computer-readable medium share the advantages of the method.

### BRIEF DESCRIPTION OF THE FIGURES

FIG. 1 depicts a flowchart, that illustrates a decentrally controlled imaging method according to a first embodiment.

FIG. 2 depicts a schematic illustration of a decentralized imaging-based data extraction system according to an embodiment.

FIG. 3 depicts a flowchart, that illustrates an automated decentrally controlled imaging method according to an embodiment.

FIG. 4 depicts a flowchart, that illustrates a decentrally controlled imaging method according to an embodiment.

FIG. 5 depicts a flowchart, that illustrates a completely automated decentrally controlled imaging method according to an embodiment.

FIG. 6 depicts a schematic illustration an artificial neural network according to an embodiment.

FIG. 7 depicts a flowchart, that illustrates a training method for training preparation of imaging on the basis of an indication selection according to an embodiment.

FIG. 8 depicts a flowchart, that illustrates a training method for training a selection of a Field of View and an image slice according to an embodiment.

FIG. 9 depicts a flowchart, that illustrates a training method for training a quality control of image data and a decision as to whether an image recording should be repeated with correspondingly amended parameters according to an embodiment.

FIG. 10 depicts a flowchart, that illustrates a training method for training a diagnosis process on the basis of image data according to an embodiment.

FIG. 11 depicts a flowchart, that illustrates a training method for training a checking process as to whether a diagnosis is sufficiently reliable or whether imaging should be carried out with contrast medium according to an embodiment.

FIG. 12 depicts a schematic illustration of a training system according to embodiment.

FIG. 13 depicts a schematic illustration of a Cloud-based decentralized image data extraction system and training data extraction system according to an embodiment.

### DETAILED DESCRIPTION

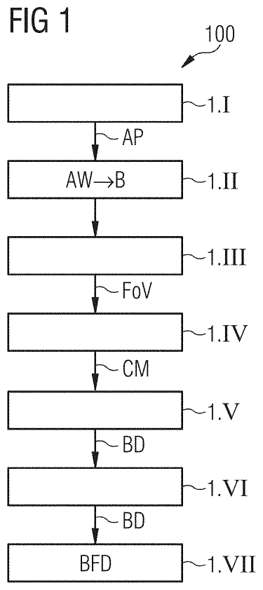

FIG. 1 depicts a flowchart 100, that illustrates an automated imaging-based data extraction method according to an embodiment. In the embodiment, a telephonically controlled imaging method is implemented. In the preliminary stage of the method illustrated in FIG. 1, a user B, for example a nurse, together with the patient, makes their way to a medical technical imaging system, in this embodiment an MR system, and switches the system on. In step 1.I, the user B presses a button, that is arranged directly on the scanning unit of the MR system. This triggers a Cloud-based application program AP, that automatically searches for an available medical technical assistant MTA via the Cloud or a data network and establishes a speech and image link between the assistant MTA and the user B. The assistant MTA includes the user interface and the entire examination space in sight via an IP camera. In step 1.II, the user B receives via the voice connection instructions AW for patient registering. Then in step 1.III, the selection of the respective field of view FOV is managed by the assistant MTA. In step 1.IV, a speech dialog occurs via further protocol parameters, such as the use of a contrast medium CM and the like. In the process the user B, in dialog with the patient P and the assistant MTA, enters appropriate protocol data via the user interface. Then in step 1.V, imaging of the field of view is carried out. The process is also monitored from a distance by the assistant MTA, for example with the aid of a network-based camera. At the end of imaging, in step 1.VI, a Cloud-based network application searches for an available radiologist, for example situated at a distant location, and the image data BD is transferred via the Internet to the available radiologists R, so, without waiting time, a diagnosis may be made using the generated image data BD. In step 1.VII, the diagnosis data BFD is transferred via the Internet to the user B and the patient P and simultaneously stored in the Cloud.

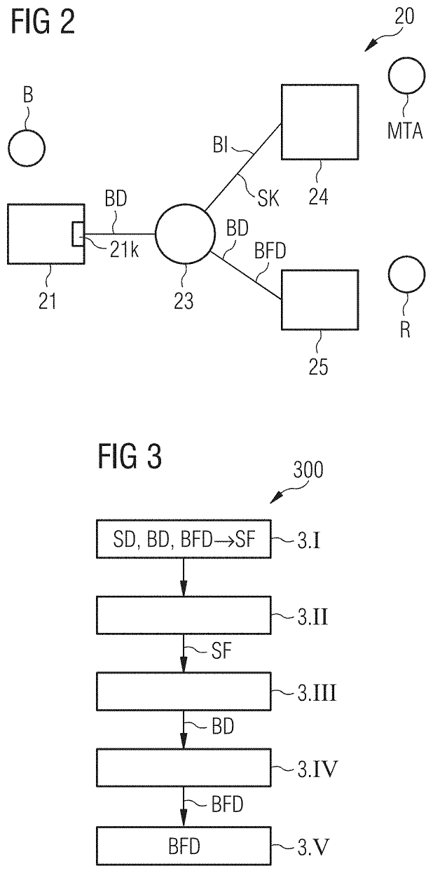

FIG. 2 depicts a schematic illustration of a Cloud-based decentralized imaging-based data extraction system 20 according to an embodiment. The data extraction system 20 includes an MR imaging device 21, in which a patient (not shown) may be placed for an imaging examination. The MR imaging device 21 is operated by a user B, that is symbolized in FIG. 2 as a circle. The user B makes a connection using a communication device 21k associated with the MR-imaging device 21 with the aid of a network application stored in a data network 23, that may be configured as a Cloud, to a specialist workstation 24, via which a medical technical assistant MTA, that is symbolized in FIG. 2 also as a circle, may conduct verbal communication SK and may obtain image information BI from the situation of the imaging in situ. The image information BI, that depicts the scenario in situ, in other words at the location of the MR imaging device 21, may be recorded for example by a camera, that is located in the space in which the MR imaging device is arranged. Part of the system 20 is also a specialist workstation 25 to which the image data BD generated by the MR imaging device 21 may be transferred via the data network 23. The diagnosis data BFD generated on the basis of this image data BD by a radiologist R, that is symbolized in FIG. 2 also by a circle, may be transferred to the specialist workstation 25 via the network 23 to the user B. Furthermore, the data BD, BFD acquired and generated during imaging and in the diagnosis may be stored in the Cloud 23 so it may be used for subsequent examinations, that possibly take place at a different location.

FIG. 3 depicts a flowchart 300, that illustrates an automated imaging-based data extraction method according to an embodiment. In this embodiment control of the imaging and of the diagnosis process occurs with the aid of artificial intelligence. In step 3.I, on the basis of the control data SD, image data BD and diagnosis data BFD generated in the method illustrated in FIG. 1, automated Cloud-based control of the imaging process and an automated diagnosis based on a neural network SF is trained. The trained neural network SF is provided via the Internet in step 3.II as an application for a user B. For example, a scan application discerns the task of a medical technical assistant MTA. To prepare and carry out an imaging process the user B now conducts in step 3.III a dialog with the scan application, without an appropriately qualified person being involved in the process. Subsequently, in step 3.IV, an automated diagnosis likewise occurs via a network application, in which again no appropriately qualified person has to be involved. In step 3.V, the user B retrieves the generated diagnosis data BFD and thereby ends the examination process.

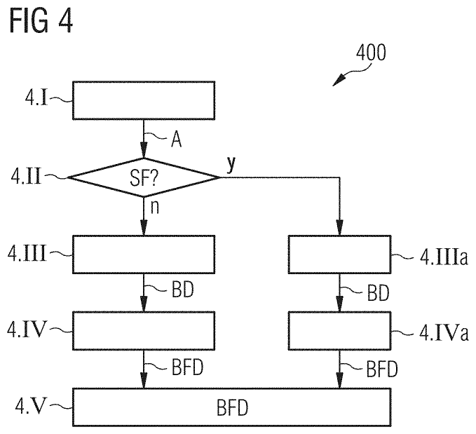

FIG. 4 illustrates a flowchart 400, that depicts an automated imaging-based data extraction method according to an embodiment. In the embodiment illustrated in FIG. 4, a fully automatic examination process is implemented. The request A for imaging and/or a diagnosis is now already transferred directly in step 4.I to a program stored in the data transmission network, that is based on an artificial neural network, without human assistant. The program classifies in step 4.II the desired processes according to whether they may be virtually or automatically processed. The classification depends for example on whether there is an application program, that is based on a trained artificial neural network SF, available for an automated application or not. The imaging is controlled and subsequently the diagnosis process as a function of the classification performed in step 4.II. As in step 4.III and 4.IV, control may be implemented virtually, in other words performed using a qualified operator operating by way of remote control or alternatively, in step 4.IIIa and in step 4.Iva, control of the imaging and the diagnosis process may be automated. Finally, in step 4.V, the diagnosis data BFD is transferred to the user B.

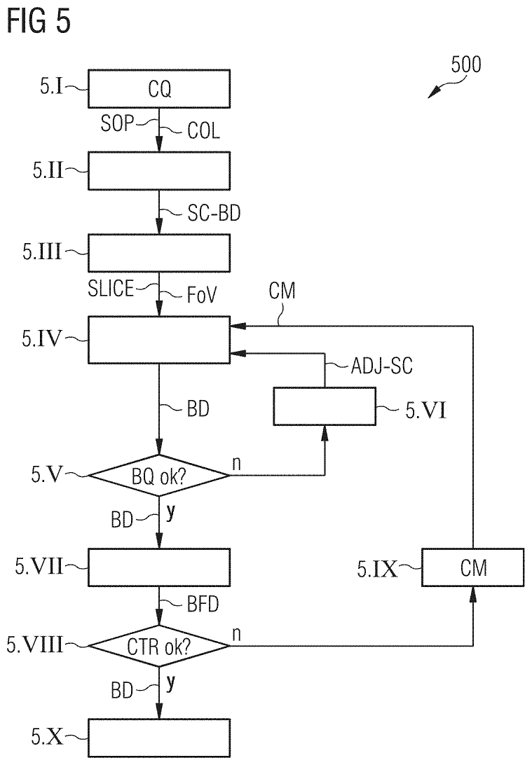

FIG. 5 depicts a flowchart 500 that illustrates a completely automated decentrally controlled imaging method according to an embodiment. The embodiment illustrated in FIG. 5 is intended to illustrate in detail control of the imaging and diagnosis.

In step 5.I, a pre-adjustment for medical technical imaging is to be determined on the basis of a clinical question CQ, that is to be responded to with the aid of imaging. For example, it should be clarified whether an organ of a patient is affected by a tumor or not. In an MR system as the medical technical imaging system, the pre-adjustment includes for example a suitable set SOP of imaging protocols and a selection COL of coils suitable for the image.

In step 5.II, a scout image SC-BD of the patient is generated to determine the position of the patient and its organs. The scout image SC-BD is used in step 5.III to define the field of view FoV, for the subsequent MR imaging and slices to be imaged SLICE.

The actual imaging process of the defined field of view FoV occurs in step 5.IV. The image data BD generated in the process is then checked in step 5.V for adequate quality BQ. If the image quality is not adequate, and this is identified in FIG. 5 by “n”, then the method skips to step 5.VI. In step 5.VI, a parameter adjustment ADJ-SC is performed and the method returns to step 5.IV, in which a renewed MR image recording occurs with adjusted parameters. If, by contrast, it is determined in step 5.V that the image quality BQ of the first image recording is adequate, and this is identified in FIG. 5 by “y”, then the method skips to step 5.VII in which the quality-checked image data BD is subject to a radiological diagnosis. The diagnosis data BFD generated in the process is then checked in step 5.VIII as to its reliability CTR. If it is determined in step 5.VIII that the diagnosis is not reliable, and this is identified in FIG. 5 by “n”, then the method skips to step 5.IX in which a contrast medium CM and its dosage is selected for renewed imaging. The method then returns to step 5.IV and imaging is repeated, etc. with the contrast medium CM. If, by contrast, it is determined in step 5.VIII that the diagnosis result BFD is sufficiently reliable, and this is identified in FIG. 5 by “n”, then the method skips to step 5.X in which the diagnosis result BFD is transferred to the user B.

The step groups 5.I, 5.II to 5.III, 5.IV to 5.VI, 5.VII and 5.VIII to 5.X illustrated in FIG. 5 may be automatically performed in each case by application of a Cloud-based computer program, whose algorithm was generated by application of an artificial neural network.

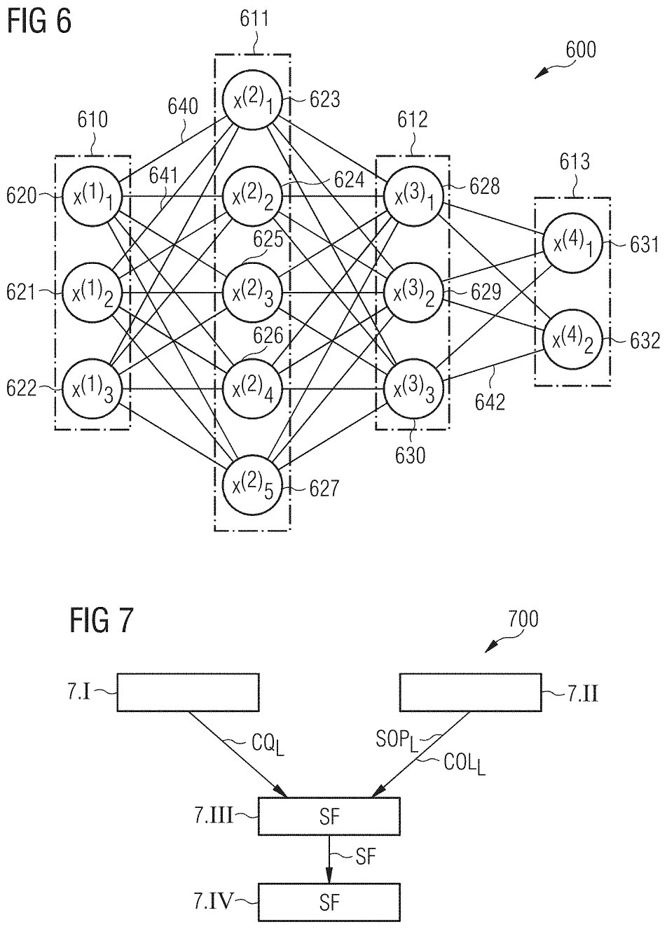

FIG. 6 schematically illustrates an artificial neural network 600. Alternative names are “neural network”, “artificial neural network” or “neuronal network”.

The artificial neural network 600 includes nodes 620, . . . , 632 and edges 640, . . . , 642. Each edge 640, . . . , 642 is a directed connection of a first node 620, . . . , 632 to a second node 620, . . . , 632. The first node 620, . . . , 632 and the second node 620, . . . , 632 are different nodes 620, . . . , 632. It is also possible that the first node 620, . . . , 632 and the second node 620, . . . , 632 are identical. For example, in FIG. 6, the edge 640 is referred to as a directed connection of the node 620 to the node 623 and the edge 642 a directed connection of the node 630 to the node 632. An edge 640, . . . , 642 from a first node 620, . . . , 632 to a second node 620, . . . , 632 is also referred to as an “incoming edge” for the second node 620, . . . , 632 and as an “outgoing edge” for the first node 620, . . . , 632.

In this embodiment the nodes 620, . . . , 632 of the artificial neural network 600 are arranged in slices 610, . . . , 613, wherein the slices may have an intrinsic sequence, that is defined by the edges 640, . . . , 642 between the nodes 620, . . . , 632. For example, the edges 640, . . . , 642 may occur only between adjacent slices or nodes. In the illustrated embodiment there is an input slice 610, that includes only nodes 620, . . . , 622 without incoming edge, an output slice 613, that includes only nodes 631, 632 without outgoing edges, and hidden slices 611, 612 between the input slice 610 and the output slice 613. In general, the number of hidden slices 611, 612 may be arbitrarily chosen.

The number of nodes 620, . . . , 622 within the input slice 610 may be associated with the number of input values of the neural network and the number of nodes 631, 632 within the output slice 613 is associated with the number of output values of the neural network.

For example, a real number may be allocated as a value to each node 620, . . . , 632 of the neural network 600. Here, x⁽ⁿ⁾ᵢ denotes the value of the i^{th} node 620, . . . , 632 of the n^{th} slice 610, . . . , 613. The values of the nodes 620, . . . , 622 of the input slice 610 match the input values of the neural network 600, the values of the nodes 631, 632 of the output slice 613 match the output values of the neural network 600. Furthermore, each edge 640, . . . , 642 may have a weight, that is a real number, for example, the weight may have a real number in the interval [−1, 1] or within the interval [0, 1]. Here, w^{(m,n)}_{i,j} denotes the weight of the edge between the i^{th} node 620, . . . , 632 of the m^{th} slice 610, . . . , 613 and the j^{th} nodes 620, . . . , 632 of the n^{th} slice 610, . . . , 613. Furthermore, the abbreviation w⁽ⁿ⁾_{i,j} is defined for the weight w^{(n, n+1)}_{i,j}.

To calculate the output values of the neural network 100, the input values are propagated by the neural network. The values of the nodes 620, . . . , 632 of the (n+1)^{th} slice 610, . . . , 613 may be calculated on the basis of the values of the nodes 620, . . . , 632 of the n^{th} slice 510, . . . , 613 by

xⱼ⁽ⁿ⁺¹⁾=f(Σᵢxᵢ⁽ⁿ⁾w_{i,j}⁽ⁿ⁾)

Here, the function f is a transfer function (a different designation is “activation function”). Known transfer functions are step functions, Sigmoid functions (for example the logistical function, the generalized logistical function, the hyperbolic tangent, the arctangent function, the error function, the smoothstep function) or rectifier functions. The transfer function is primarily used for normalization purposes.

The values x⁽ⁿ⁾ᵢ are propagated slice-wise by the neural network, wherein the values of the first hidden slice 611 may be calculated on the basis of the values of the input slice 610 of the neural network and wherein the values of the second hidden slice 612 may be calculated on the basis of the values of the first hidden slice 611.

To define the values w^{(m,n)}_{i,j} for the edges, the neural network 600 is trained using training data. The training data includes training input data and training output data (designated by yᵢ). For a training step, the neural network 600 is applied to the training input data to generate calculated output data. The training data and the calculated output data have a number of values, whose number is equal to the number of the nodes of the output slice.

A comparison between the calculated output data and the training data is used to recursively adjust the weights within the neural network 600 (a backpropagation algorithm is applied for this). The weights are changed according to the following formula:

w′_{i,j}ⁿ=w_{i,j}⁽ⁿ⁾−γδⱼ⁽ⁿ⁾·xᵢ⁽ⁿ⁾,

where γ is a learning rate and the numbers δ⁽ⁿ⁾ⱼ may be recursively calculated as:

δⱼ⁽ⁿ⁾=(Σₖδₖ⁽ⁿ⁺¹⁾·w_{j,k}⁽ⁿ⁺1)·f′(Σᵢx_{i,j}⁽ⁿ⁾·w_{i,j}⁽ⁿ⁾)

Based on δ⁽ⁿ⁺¹⁾ⱼ, if the (n+1)^{th} slice is not the output slice, and

δⱼ⁽ⁿ⁾=(xₖ⁽ⁿ⁺¹⁾)·f′(Σᵢx_{i,j}⁽ⁿ⁾·w_{i,j}⁽ⁿ⁾),

if the (n+1)^{th} slice is the output slice 613, where f′ is the first derivation of the activation function and y⁽ⁿ⁺¹⁾ⱼ is the comparison training value for the j^{th} nodes of the output slice 613.

FIG. 7 depicts a flowchart 700, that illustrates a training method for training preparation of imaging on the basis of an indication selection. In step 7.I, labeled question data CQL is read from a training database. Furthermore, in step 7.II, protocol data SOP_{L} and data for coil sets COL_{L} are read from a training database. The training data is then used in step 7.III for training a neural network SF. The trained neural network SF is provided in step 7.IV for automated imaging preparation.

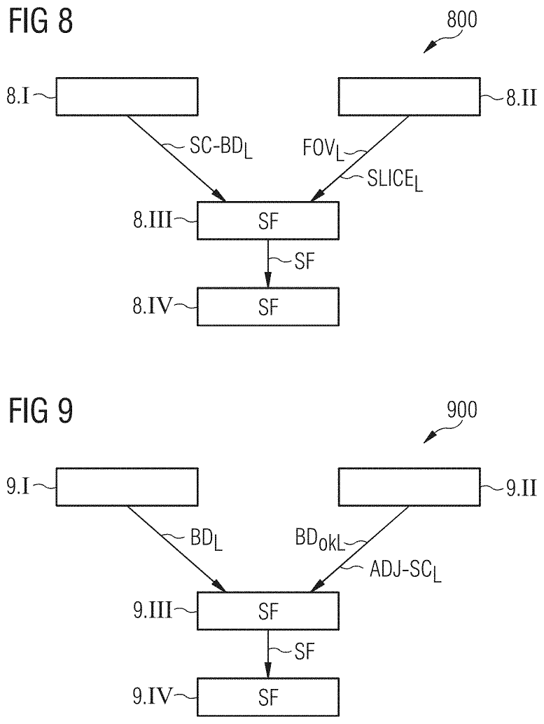

FIG. 8 depicts a flowchart 800, that illustrates a training method for training what is known as an auto-align process of imaging. In step 8.I, labeled scout image data SD-BD_{L} is read from a training database. In addition, in step 8.II, labeled data FoV_{L} for definition of the region to be imaged and labeled data SLICE_{L} for definition of slices to be scanned during subsequent imaging is read from the training database. The training data SD-BD_{L}, FoV_{L}, SLICE_{L} is used in step 8.III for training a neural network SF. The trained neural network SF is provided in step 8.IV for an automated auto-align process.

FIG. 9 depicts a flowchart 900, that illustrates a training method for training control of the actual imaging process. In step 9.I, labeled image data BD_{L} is read from a training database. In addition, in step 9.II, labeled data BD_{okL}, that provides information as to whether the quality of the labeled image data BD_{L} is sufficient. In step 9.II, labeled data ADJ-SC_{L} is also read from the training database, that provides information about an adjustment of parameters of imaging to be repeated. The data is required if the image quality of the image data BD_{L} of the first imaging is classed as inadequate. The training data BD_{L}, BD_{okL}, ADJ-SC_{L} is then used in step 9.III for training a neural network SF. The trained neural network SF is provided in step 9.IV for an automated imaging process.

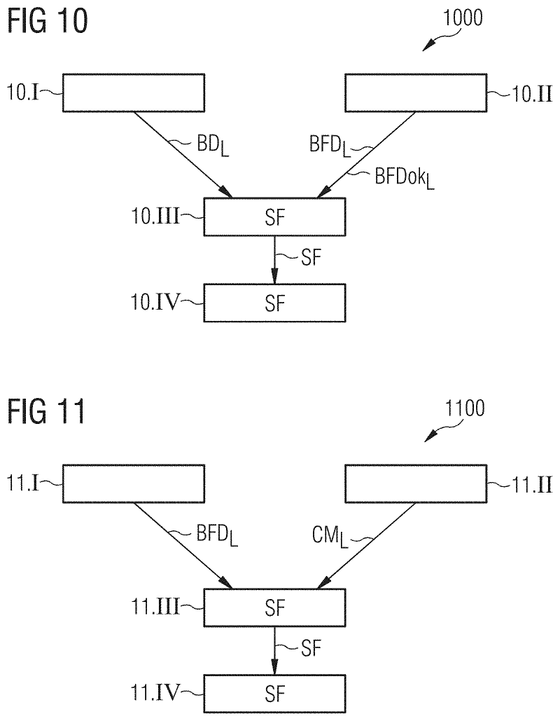

FIG. 10 depicts a flowchart 1000, that illustrates a training method for training an automated diagnosis process. In step 10.I, labeled image data BD_{L} is read from a training database. In addition, in step 10.II, labeled diagnosis data BFD_{L} is read from the training database. Furthermore, in step 10.II, information BFD-ok_{L} is also read out as to whether the diagnosis result is sufficiently reliable or not. The training data BD_{L}, BFD_{L}, BFDok_{L} is used in step 10.III for training a neural network SF. The trained neural network SF is provided in step 10.IV for an automated diagnosis process.

FIG. 11 depicts a flowchart 1100, that illustrates a training method for training an automated refinement process. In step 11.I, labeled diagnosis data BFD_{L} is read from a training database. In addition, in step 11.II, labeled data CM_{L} is read from the training database that gives information about which contrast medium, and at which dose, should be used for renewed refined imaging.

The training data BFD_{L}, CM_{L} is used in step 11.III for training a neural network SF. The trained neural network SF is provided in step 11.IV for an automated diagnosis process.

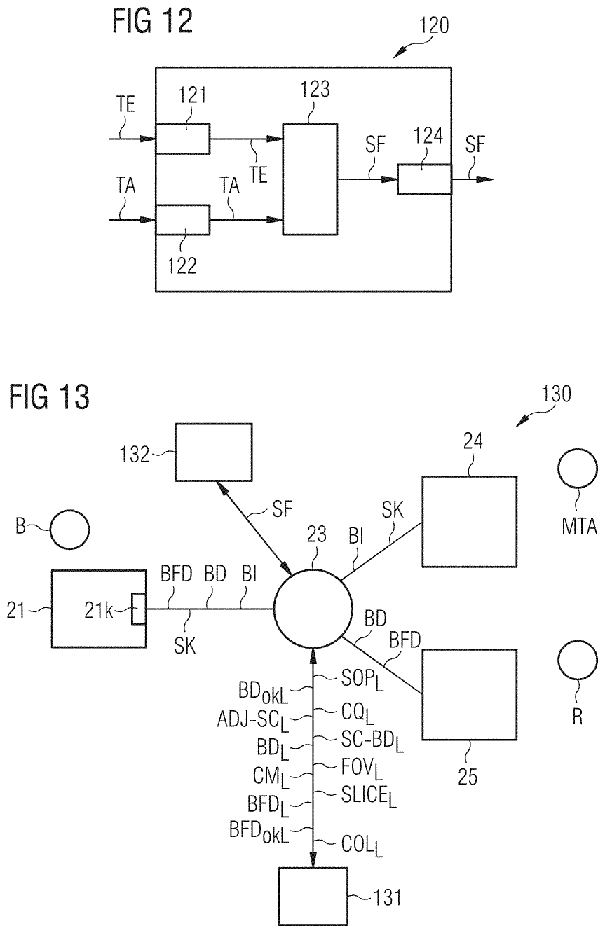

FIG. 12 illustrates a schematic representation of a training system 120 according to one embodiment. The training system 120 includes a first training interface 121 for receiving input training data TE. The input training data may include, for example, the labeled question data CQ_{L} described in FIG. 7 to FIG. 11, labeled scout image data SD-BD_{L}, labeled image data BD_{L} or labeled diagnosis data BFD_{L}. A second training interface 122 for receiving output training data TA is also part of the training system 120. The output training data TA may include, for example, the labeled protocol data SOP_{L} described in FIG. 7 to FIG. 11 and data for coil sets COL_{L}, labeled data FOV_{L} for defining the region to be imaged or labeled data SLICE_{L} for defining slices to be scanned during subsequent imaging, labeled data BD_{okL}, that provides information about whether the quality of the labeled image data BD_{L} is adequate, labeled data ADJ-SC_{L}, that provides information about an adjustment of parameters of imaging to be repeated, or information BFDok_{L} about whether the diagnosis result is sufficiently reliable or not, and labeled data CM_{L}, that provides information about which contrast medium, and at which dose, should be used for renewed refined imaging. A training arithmetic unit 123 for training a function SF for automated image control on the basis of the input training data TE and the output training data TA is also part of the training system 120. The training system 120 also includes a third training interface 124 for providing the training function SF for an automated system.

FIG. 13 depicts a schematic illustration of a Cloud-based decentralized image data extraction system 130 according to an embodiment. In the embodiment shown in FIG. 13, in addition to the access points 24, 25 for the medical technical assistant MTA and the radiologists R, there are also artificial intelligences based on neural networks SF present, that accompany imaging or make the diagnosis as an alternative to the human specialists. For training the software-based services, there are corresponding training programs in the Cloud 23, that access the protocol data, image data and diagnosis data stored in the Cloud. The system 130 described in FIG. 13 may also be used as the training data extraction system. The training data manually generated by the medical technical assistant MTA and the radiologists R generated is stored in a training database 131. For training a control function or an artificial neural network SF, the data may be read from the training database 131. The artificial neural networks SF generated in the training process is stored in a network database 132 and may be used by the Cloud 23 at any time for a simulation of a medical technical assistant MTA or a radiologist R.

A neural network may also be trained for the management of the system shown in FIG. 13. A neural network may classify tasks relating to imaging into tasks suitable for a virtual qualified operator MTA, R or on automated system SF. The tasks classified as being automatically processible may then be processed by the neural networks that already exist in the database 132 or computer programs based thereon. The classification may occur, for example, on the basis of the number of training data available for training a neural network.

Finally, reference is made once again to the fact that the above-described methods and devices are merely embodiments and that the embodiments may be varied by a person skilled in the art without departing from the scope as far as it is specified by the claims. For the sake of completeness reference is also made to the fact that use of the indefinite article “a” or “an” does not preclude the relevant features from also being present several times. Similarly, the term “unit” does not preclude this from including a plurality of components, that may be spatially distributed also.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for decentrally controlled imaging, the method comprising:
selecting a control entity that is connected by a data transmission network to a communication device of a medical technical imaging system;
initializing a network-based communications link between the communication device of the medical technical imaging system and the control entity; and
remotely controlling an image recording process of a field of view of a patient by the control entity via the network-based communications link.

2. The method of claim 1, wherein the control entity comprises a specialist workstation configured to acquire and transfer control data via the network-based communications link for logging creation.

3. The method of claim 2, wherein the image recording process is remotely controlled on the basis of a dialogue, conducted via the network-based communications link, between a qualified operator at the specialist workstation and a user situated at a location of the medical technical imaging system.

4. The method of claim 1, further comprising:
selecting one specialist workstation that is connected to the communication device of the medical technical imaging system from a plurality of specialist workstations that includes at least one specialist workstation for a qualified operator of a plurality of qualified operators;
transferring image data generated during the image recording process to the selected specialist workstation; and
outputting diagnosis data from the specialist workstation.

5. The method of claim 4, wherein at least one of the qualified operators is simulated by an automated control system trained with training data.

6. The method of claim 5, wherein use of the trained automated control system is started as a function of whether a sufficient number of training data sets for the training process of the respective automated control system is available.

7. The method of claim 6, wherein tasks relating to imaging are classified into tasks, that are suitable for a virtual qualified operator or an automated system on the basis of artificial intelligence.

8. A system for decentralized image data extraction, the system comprising:
a medical technical imaging system;
a switching unit configured to determine and select a control entity that is connected via a data transmission network to a communication device of the medical technical imaging system;
an initialization device configured to initialize a network-based communications link between the communication device of the medical technical imaging system and the control entity; and
the control entity configured to remotely control an image recording process of a field of view of a patient via the network-based communications link.

9. The system of claim 8, wherein the medical technical imaging system comprises a CT system or an MR system.

10. The system of claim 8, wherein the switching unit is further configured to determine and select a specialist workstation for a qualified operator that is connected to the communication device of the medical technical imaging system from a plurality of specialist workstations, wherein the initialization device is configured to initialize a network-based communications link between the communication device of the medical technical imaging system and the selected specialist workstation for transferring image data generated during the image recording process to the selected specialist workstation, the system further comprising an output interface for outputting diagnosis data from the specialist workstation.

11. A method for the extraction of training data using an automated image data extraction system, the method comprising:
selecting a control entity that is connected by a data transmission network to a communication device of a medical technical imaging system;
initializing a network-based communications link between the communication device of the medical technical imaging system and the control entity;
remotely controlling an image recording process of a field of view of a patient by the control entity via the network-based communications link;
selecting one specialist workstation that is connected to the communication device of the medical technical imaging system from a plurality of specialist workstations that includes at least one specialist workstation for a qualified operator of a plurality of qualified operators;
transferring image data generated during the image recording process to the selected specialist workstation;
outputting diagnosis data from the specialist workstation; and
extracting input training data and output training data from the image data and diagnosis data.

12. The method as claimed in claim 11, wherein the input training data comprises at least one of the following data types: patient data, examination data, technical data relating to the medical technical imaging system, scout image data, image data, and wherein output training data comprises at least one of the following data types: control data, which was generated by the qualified operator, a number of an organ program, alignment data, an instruction to repeat the image recording, adjusted parameter data for a repeated image recording, or diagnosis data.

13. A method for providing a trained automated control system, the method comprising:
receiving labeled input training data;
receiving labeled output training data, wherein the output training data is connected with the input training data;
training a function on the basis of the input training data and the output training data;
providing the trained function for an automated control system.

14. The method of claim 13 wherein further comprising:
selecting a control entity that is connected by a data transmission network to a communication device of a medical technical imaging system;
initializing a network-based communications link between the communication device of the medical technical imaging system and the control entity;
remotely controlling an image recording process of a field of view of a patient by the control entity via the network-based communications link;
selecting the trained automated control system that is connected to the communication device of the medical technical imaging system;
transferring image data generated during the image recording process to the trained automated control system; and
outputting diagnosis data from the trained automated control system.

15. The method of claim 14, wherein the medical technical imaging system comprises a CT system or an MR system.

16. The method of claim 14, wherein the labeled input training data comprises at least one of the following data types: patient data, examination data, technical data relating to the medical technical imaging system, scout image data, image data, and wherein labeled output training data comprises at least one of the following data types: control data, which was generated by a qualified operator, a number of an organ program, alignment data, an instruction to repeat the image recording, adjusted parameter data for a repeated image recording, or diagnosis data.
